(19) United States
(12) Patent Application Publication
(10) Pub. No.: US 2021/0309994 A1
(43) Pub. Date: Oct. 7, 2021

(54) INLINE INDEX BASED ON ILLUMINA SEQUENCING, AND DNA LIBRARY LABELED THEREBY AND METHOD FOR CONSTRUCTING SAME

(71) Applicant: Shanghai Ocean University, Shanghai (CN)

(72) Inventors: Chenhong LI, Shanghai (CN); Ying WANG, Shanghai (CN); Hao YUAN, Shanghai (CN)

(21) Appl. No.: 16/693,253

(22) Filed: Nov. 23, 2019

(30) Foreign Application Priority Data
Nov. 23, 2018 (CN) 201811406204.X

(51) Int. Cl. C12N 15/10

(52) U.S. Cl. CPC C12N 15/1068

(57) ABSTRACT

Disclosed are an inline index based on Illumina sequencing, a DNA library labeled thereby and a method of constructing the same, where the method includes the steps of: breaking DNA sequence of a sample; repairing a flat end; ligating an inline index adapter to an end of the sequence; repairing a gap of the sequence and extending the sequence; and subjecting the sequence to PCR amplification to construct the DNA library. The inline index is a 6-bp DNA sequence free of ‘AAA’, ‘ACA’, ‘CCC’, ‘CAC’, ‘GGG’, ‘GTG’, ‘TTT’ and ‘TGT’. In addition, the inline index also has a minimum editing length equal to or less than 3, and includes bases of different laser colors, where the laser colors of adjacent bases are different.

### REFERENCE TO AN ELECTRONIC SEQUENCE LISTING

The contents of the electronic sequence listing (Untitled ST25.txt; Size: 18,000 bytes; and Date of Creation: Aug. 18, 2020) is herein incorporated by reference in its entirety.

### CROSS-REFERENCE TO RELATED APPLICATIONS

This application claims the benefit of priority from Chinese Patent Application No. 201811406204.X, filed on Nov. 23, 2018. The content of the aforementioned application, including any intervening amendments thereto, is incorporated herein by reference.

### TECHNICAL FIELD

This application relates to molecular labeling, particularly to the labeling of DNA library, and more particularly to an inline index based on Illumina sequencing, a DNA library labeled thereby and a method for constructing the same.

### BACKGROUND

Currently, the construction method of a universal DNA library involving Illumina sequencing generally includes the steps of: breaking the DNA sequence of a biological sample; repairing the flat end; ligating adapters of IS7 and IS8 primer recognition sites; filling the gap between respective adapters and the sequence; introducing a barcode to the sequence during the indexing PCR to differentiate the samples; and sorting the sequencing data according to the barcode. However, in the above method, the barcode is introduced at the end of the entire experiment, so that if the samples are mutually contaminated before, it is impossible to determine which sample the data is derived from.

In the prior art, Chinese Patent Application No. 107502607A discloses a method involving the molecular barcode labeling, library construction and sequencing for mRNA from a large number of tissues and cells, which can be applied to the labeling during the reverse transcription of mRNA and the synthesis of cDNA. However, this method is not suitable for the construction of DNA library. Chinese Patent No. 104573407B discloses a method of searching a species-specific endogenous barcode and an application thereof in the pooled sequencing for multiple samples. In this method, the PCR products are labeled using gene splicing by overlap extension PCR (SOE PCR) according to the differences in the endogenous DNA from individual samples, which does not involve the DNA library construction and the barcode labeling. At present, the cross contamination is frequently observed among DNA samples, greatly affecting the subsequent assembly and analysis of data.

### SUMMARY

An object of this application is to provide an inline index based on Illumina sequencing, and a DNA library labeled thereby and a method for constructing the same to overcome the defects in the prior art. In the library construction method provided herein, an inline index adapter is introduced in the initial stage, and the diverse DNA samples can be accurately distinguished by labeling the constructed DNA library with the inline index. Compared to the conventional molecular index technique, this application can not only correct the contaminated data, but also greatly increase the number of index combinations, facilitating the differentiation of more samples and lowering the cost.

The technical solutions of this application are described as follows. In a first aspect, this application provides an inline index based on Illumina sequencing, wherein the inline index is a 6-bp DNA sequence, and the inline index has the following characteristics:

i) it has a minimum editing length of 3 bp or less;

ii) it comprises fragments of different bases;

iii) it comprises bases of different laser colors, wherein adjacent bases are different in laser color; and

iv) it is free of ‘AAA’, ‘ACA’, ‘CCC’, ‘CAC’, ‘GGG’, ‘GTG’, ‘TTT’ and ‘TGT’.

In an embodiment, the inline index comprises an IS1 inline index, an IS2 inline index and an IS3 inline index corresponding to the IS1 inline index and IS2 inline index, where the IS3 inline index is partially complementary to the IS1 inline index and the IS2 inline index.

In an embodiment, the IS1 inline index comprises IS1 sequence and IS3X′ sequence partially complementary to the IS1 sequence; and the IS1 sequence and the IS3X′ sequence are ligated by cooling from 95° C. to 12° C. at a rate of 0.1° C/s.

In an embodiment, the IS2 inline index comprises IS2 sequence and IS3Y′ sequence partially complementary to the IS2 sequence; and the IS2 sequence and the IS3Y′ sequence are ligated by cooling from 95° C. to 12° C. at a rate of 0.1° C/s.

In a second aspect, this application further provides a method of constructing a DNA library labeled by the above inline index, which specifically comprises:

(1) breaking a DNA sequence of a biological sample to obtain a DNA fragment;

(2) repairing a blunt endof the DNA fragment;

(3) ligating an adapter of the IS1 inline index to a 5′ end of the DNA fragment; and ligating an adapter of the IS2 inline index to a 3′ end of the DNA fragment; wherein the adapter of the IS1 inline index is inside a binding site of a primer IS7, and the adapter of the IS2 inline index is inside a binding site of a primer IS8;

(4) repairing a gap of the DNA fragment and extending the repaired DNA fragment; and

(5) subjecting the extended DNA fragment to PCR amplification in the use of the primers IS7 and IS8 to produce the DNA library.

This application has the following beneficial effects.

In the conventional methods for constructing a DNA library, the cleaning process is required to be repeated, which will easily cause cross contamination among the samples or contamination derived from exogenous DNA. In addition, due to the high sensitivity of the biological probe, a low concentration of exogenous DNA may also be captured and sequenced. This application introduces an inline index to label the DNA in advance, which greatly reduce the occurrence that the contamination data is recognized, significantly improving the problem that cross contamination frequently occurs among the samples during the construction of DNA library and the enrichment of genes. Moreover, after labeled with the inline index provided herein, the DNA samples may be mixed for the gene enrichment and sequencing, greatly reducing the complexity in the operation of gene enrichment.

### BRIEF DESCRIPTION OF THE DRAWINGS

FIG. 1 schematically shows the preparations of IS1 and IS2 inline indexes; where the IS1 inline index is formed by the combination of IS1 and IS3X′ sequences through the cooling from 95° C. to 12° C. at a rate of 0.1° C/s; and the IS2 inline index is formed by the combination of IS2 and IS3Y′ sequences through the cooling from 95° C. to 12° C. at a rate of 0.1° C./s.

FIG. 2 schematically shows the construction of DNA library, where the construction process sequentially includes: repairing a blunt endof a DNA sequence; ligating inline index adapters at both ends; repairing the gap and extending the DNA; and subjecting the DNA sequence to PCR amplification in the use of primers IS7 and IS8 to produce the DNA library since the binding sites of primer IS7 and primer IS8 are located at the outermost side of the adapter sequences of IS1 and IS2, respectively. During the gene enrichment, a specific probe is used to capture a target fragment from the DNA library, and the binding sites of primer IS4 and the index primer for sequencing are located outside the primer IS7 and the primer IS8, respectively. After the final target fragment is obtained, the DNA sequence is subjected to index PCR amplification and Illumina sequencing. Optionally, the DNA library can be amplified using primer IS4 and the index primer and directly sequenced to obtain genomic information.

### DETAILED DESCRIPTION OF EMBODIMENTS

This application will be described in detail below with reference to the drawings and embodiments, but these embodiments are not intended to limit the application.

### EXAMPLE 1

Inline indexes were respectively synthesized according to the DNA sequences listed in Table 1, and the process was specifically described as follows.

An Oligo Hybridization Buffer consisting of 1 mL of 5 M NaCl, 100 μL of 1 M Tris-HCl (pH 8.0), 20 μL of 0.5 M EDTA (pH 8.0) and 8.88 mL of H₂O was prepared in advance.

10 μL of 500 μM IS1_adapter_P5.F, 10 μL of 500 μM IS3 adapter P5+P7.R, 10 μL of 10x Oligo Hybridization Buffer and 70 μL of H20 were mixed for the preparation of a double-strand adapter.

10 μL of 500 μM IS2 adapter P7.F, 10 μL of 500 μM IS3 adapter P5+P7.R, 10 μL of 10× Oligo Hybridization Buffer and 70 μL of H20 were mixed for the preparation of a double-strand adapter.

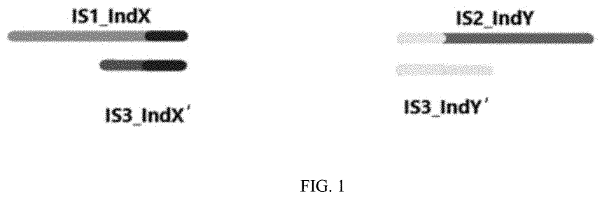

The above two reaction mixtures were respectively reacted at 95° C. in a PCR instrument for 10 s and then cooled to 12° C. at a rate of 0.1° C/s. Then the two reaction mixtures were respectively dispensed at 20 μL, per tube, and the tubes were numbered and stored at −20° C. for use. The resulting double-strand adapters were shown in FIG. 1.

TABLE 1
Information of inline index

| | Name | Sequence | Name | Sequence |
|---|---|---|---|---|
| TCTGCC | IS1 Ind1 | SEQ ID NO: 1 | IS3 Ind1 | SEQ ID NO: |
| | | | | 49 |
| GTCTCT | IS1 Ind2 | SEQ ID NO: 2 | IS3 Ind2 | SEQ ID NO: |
| | | | | 50 |
| ATATTG | IS1 Ind3 | SEQ ID NO: 3 | IS3 Ind3 | SEQ ID NO: |
| | | | | 51 |
| TGGAAG | IS1 Ind4 | SEQ ID NO: 4 | IS3 Ind4 | SEQ ID NO: |
| | | | | 52 |
| TCTAGT | IS1 Ind5 | SEQ ID NO: 5 | IS3 Ind5 | SEQ ID NO: |
| | | | | 53 |
| AGAGTA | IS1 Ind6 | SEQ ID NO: 6 | IS3 Ind6 | SEQ ID NO: |
| | | | | 54 |
| GGCCAA | IS1 Ind7 | SEQ ID NO: 7 | IS3 Ind7 | SEQ ID NO: |
| | | | | 55 |
| TATCTC | IS1 Ind8 | SEQ ID NO: 8 | IS3 Ind8 | SEQ ID NO: |
| | | | | 56 |
| TTATGC | IS1 Ind9 | SEQ ID NO: 9 | IS3 Ind9 | SEQ ID NO: |
| | | | | 57 |
| AGTTGG | IS1 Ind10 | SEQ ID NO: 10 | IS3 Ind10 | SEQ ID NO: |
| | | | | 58 |
| GTCAAG | IS1 Ind1l | SEQ ID NO: 11 | IS3 Ind11 | SEQ ID NO: |
| | | | | 59 |
| CAGCAA | IS1 Ind12 | SEQ ID NO: 12 | IS3 Ind12 | SEQ ID NO: |
| | | | | 60 |
| TCGCCG | IS1 Ind13 | SEQ ID NO: 13 | IS3 Ind13 | SEQ ID NO: |
| | | | | 61 |
| CTAAGA | IS1 Ind14 | SEQ ID NO: 14 | IS3 Ind14 | SEQ ID NO: |
| | | | | 62 |
| CCGCTT | IS1 Ind15 | SEQ ID NO: 15 | IS3 Ind15 | SEQ ID NO: |
| | | | | 63 |
| AAGTTA | IS1 Ind16 | SEQ ID NO: 16 | IS3 Ind16 | SEQ ID NO: |
| | | | | 64 |
| GGTACC | IS1 Ind17 | SEQ ID NO: 17 | IS3 Ind17 | SEQ ID NO: |
| | | | | 65 |
| CCAGGT | IS1 Ind18 | SEQ ID NO: 18 | IS3 Ind18 | SEQ ID NO: |
| | | | | 66 |
| AATCGA | IS1 Ind19 | SEQ ID NO: 19 | IS3 Ind19 | SEQ ID NO: |
| | | | | 67 |
| AACGCA | IS1 Ind20 | SEQ ID NO: 20 | IS3 Ind20 | SEQ ID NO: |
| | | | | 68 |
| GACGAC | IS1 Ind21 | SEQ ID NO: 21 | IS3 Ind21 | SEQ ID NO: |
| | | | | 69 |
| CGCGCT | IS1 Ind22 | SEQ ID NO: 22 | IS3 Ind22 | SEQ ID NO: |
| | | | | 70 |
| CCGTAG | IS1 Ind23 | SEQ ID NO: 23 | IS3 Ind23 | SEQ ID NO: |
| | | | | 71 |
| GTAATC | IS1 Ind24 | SEQ ID NO: 24 | IS3 Ind24 | SEQ ID NO: |
| | | | | 72 |
| GACCTT | IS2 Ind25 | SEQ ID NO: 25 | IS3 Ind25 | SEQ ID NO: |
| | | | | 73 |
| TCATAA | IS2 Ind26 | SEQ ID NO: 26 | IS3 Ind26 | SEQ ID NO: |
| | | | | 74 |
| CAAGAG | IS2 Ind27 | SEQ ID NO: 27 | IS3 Ind27 | SEQ ID NO: |
| | | | | 75 |
| CGATCA | IS2 Ind28 | SEQ ID NO: 28 | IS3 Ind28 | SEQ ID NO: |
| | | | | 76 |
| TTGATT | IS2 Ind29 | SEQ ID NO: 29 | IS3 Ind29 | SEQ ID NO: |
| | | | | 77 |
| TCCGAG | IS2 Ind30 | SEQ ID NO: 30 | IS3 Ind30 | SEQ ID NO: |
| | | | | 78 |
| CCTGAA | IS2 Ind31 | SEQ ID NO: 31 | IS3 Ind31 | SEQ ID NO: |
| | | | | 79 |
| ATTCTT | IS2 Ind32 | SEQ ID NO: 32 | IS3 Ind32 | SEQ ID NO: |
| | | | | 80 |
| GCGACT | IS2 Ind33 | SEQ ID NO: 33 | IS3 Ind33 | SEQ ID NO: |
| | | | | 81 |
| GGCTTC | IS2 Ind34 | SEQ ID NO: 34 | IS3 Ind34 | SEQ ID NO: |
| | | | | 82 |
| AATACG | IS2 Ind35 | SEQ ID NO: 35 | IS3 Ind35 | SEQ ID NO: |
| | | | | 83 |
| TACGGT | IS2 Ind36 | SEQ ID NO: 36 | IS3 Ind36 | SEQ ID NO: |
| | | | | 84 |
| ACCGTC | IS2 Ind37 | SEQ ID NO: 37 | IS3 Ind37 | SEQ ID NO: |
| | | | | 85 |
| AGAAGC | IS2 Ind38 | SEQ ID NO: 38 | IS3 Ind38 | SEQ ID NO: |
| | | | | 86 |
| CATAGC | IS2 Ind39 | SEQ ID NO: 39 | IS3 Ind39 | SEQ ID NO: |
| | | | | 87 |
| AGGCTC | IS2 Ind40 | SEQ ID NO: 40 | IS3 Ind40 | SEQ ID NO: |
| | | | | 88 |
| CTGCGG | IS2 Ind41 | SEQ ID NO: 41 | IS3 Ind41 | SEQ ID NO: |
| | | | | 89 |
| CTCGGC | IS2 Ind42 | SEQ ID NO: 42 | IS3 Ind42 | SEQ ID NO: |
| | | | | 90 |
| GATTAG | IS2 Ind43 | SEQ ID NO: 43 | IS3 Ind43 | SEQ ID NO: |
| | | | | 91 |
| AGATAT | IS2 Ind44 | SEQ ID NO: 44 | IS3 Ind44 | SEQ ID NO: |
| | | | | 92 |
| TGGTCC | IS2 Ind45 | SEQ ID NO: 45 | IS3 Ind45 | SEQ ID NO: |
| | | | | 93 |
| GTTCCG | IS2 Ind46 | SEQ ID NO: 46 | IS3 Ind46 | SEQ ID NO: |
| | | | | 94 |
| GTACGT | IS2 Ind47 | SEQ ID NO: 47 | IS3 Ind47 | SEQ ID NO: |
| | | | | 95 |
| AAGAAC | IS2 Ind48 | SEQ ID NO: 48 | IS3 Ind48 | SEQ ID NO: |
| | | | | 96 |

* represents modification with PTO.

### EXAMPLE 2

The DNA library was constructed as follows.

A DNA sequence of a biological sample was fractured into multiple DNA fragments with a length of about 250-500 bp using a Covaris M220 Ultrasonic Processor (Covaris, Inc. Massachusetts USA), where the fracturing process was programmed as follows: running for 90 s (50 peak power, 25 duty factor, 200 cycles/burst) followed by a pause for 60 s; and running for 90 s (50 peak power, 25 duty factor, 200 cycles/burst). 35 μL of MagNA beads were added to a 270 μL centrifuge tube, and then the centrifuge tube was allowed to stand on a magnetic plate for 1 min. The supernatant was removed, and the MagNA beads were dried, added with 60 μL of the DNA sample and 54 μL of MagNA beads Buffer and mixed uniformly. Positive and negative controls were prepared at the same time. The centrifuge tube was placed at room temperature for 10 min and then transferred to the magnetic plate. The supernatant was removed, and the beads were added with 186 μL 70% ethanol and placed for 1 min. Then the ethanol was removed, and the process of adding and removing ethanol was repeated once. The centrifuge tube was placed for 5 min with the cover opened to allow the residual ethanol to volatize.

20 μL of a first mixture, consisting of 2.2 μL of 10× Buffer Tango, 0.22 μL of 1 × dNTPs (10 mM each), 0.22 μL of 100 mM ATP, 1.1 μL of T4 polynucleotide kinase (10 U/μL), 0.44 μL of T4 DNA polymerase (5 U/μL) and 17.82 μL of H₂O, was added to the centrifuge containing the dried MagNA beads under an ice bath. The reaction mixture was mixed uniformly, and then the centrifuge tube was transferred to a PCR instrument to repair a sticky end of the DNA sample, where the reparation was programmed as follows: 25° C. for 15 min and 12° C. for 5 min. Then the centrifuge tube was taken out and added with 18 μL of MagNA beads Buffer. The reaction mixture was mixed uniformly and allowed to stand on the magnetic plate for 5 min. The supernatant was removed, and the beads were added with 186 μL of 70% ethanol and allowed to stand at room temperature for 1 min. Then the ethanol was removed, and the process of adding and removing ethanol was repeated once. The centrifuge tube was allowed to stand for 5 min with the cover opened to allow the residual ethanol to volatize. 38 μL of a second mixture, consisting of 4.4 μL of 10×T4DNA ligase buffer, 4.4 μL of 50% PEG-4000, 1.1 μL of T4 DNA ligase (5U/μL) and 31.9 μL of H₂O, was prepared and added to the centrifuge tube containing the dried MagNA beads under an ice bath. Inline indexes of the sample, positive control and negative control were respectively added. Then the centrifuge tube was placed in the PCR instrument and the operation was performed at 22° C. for 30 min. After that, the centrifuge tube was taken out and added with 36 μL of MagNA beads Buffer. The reaction mixture was fully mixed and allowed to stand on the magnetic board for 5 min. The supernatant was removed, and the beads were added with 186 μL of 70% ethanol and allowed to stand at room temperature for 1 min. Then the ethanol was removed, and the process of adding and removing ethanol was repeated once. The centrifuge tube was allowed to stand for 5 min with the cover opened to allow the residual ethanol to volatize.

Bsm polymerase was introduced to extend the sequence to fill the gap between the adapter and the sequence after the addition of the inline index. 40 μL of the Bsm polymerase mixture, consisting of 4.4 μL of 10× Bsm buffer, 1.1 μL of dNTPs (10 mM each), Bsm polymerase, 1.65 μL of large fragment (8U/μL) and 36.85 μL of H₂O, was prepared and added to the centrifuge tube containing the dried MAGNA beads under an ice bath. Then the centrifuge tube was transferred to the PCR instrument and the operation was performed at 37° C. for 20 min. After that, the centrifuge tube was taken out and added with 36 μL of MagNA beads Buffer. The reaction mixture was fully mixed and allowed to stand on the magnetic board for 5 min. The supernatant was removed, and the beads were added with 186 μL of 70% ethanol and allowed to stand at room temperature for 1 min. Then the ethanol was removed, and the process of adding and removing ethanol was repeated once. The centrifuge tube was allowed to stand for 5 min with the cover opened to allow the residual ethanol to volatize. The beads were added with 35 μL of TE Buffer, and the mixture was transferred to another centrifuge tube (named as lib) and stored at −20° C. for use.

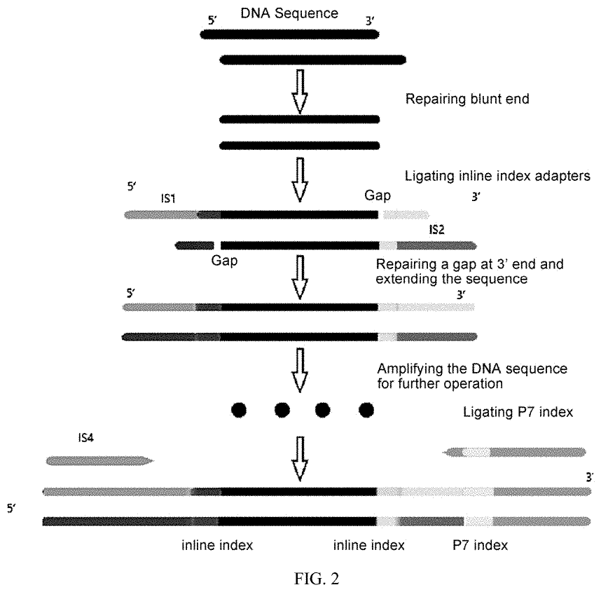

Referring to FIG. 2, through the above processes of repairing the blunt end of the DNA sequence, adding the inline index adapters at both ends, repairing the gap and extending the DNA sequence, the DNA sequence was further subjected to PCR amplification to construct the DNA library in the use of primers IS7 and IS8 since the binding sites of the primers IS7 and IS8 were respectively located at the outmost side of the adapters of IS1 and IS2. In addition, during the gene enrichment, a specific probe was used to capture a target fragment from the DNA library, and after the target fragment was obtained, the DNA sequence can be subjected to the index PCR amplification and Illumina sequencing since the binding sites of the primer IS4 and the index primer were respectively located outside the primers IS7 and IS8. Moreover, the DNA library can be amplified and directly sequenced to obtain the genomic information in the use of the primer IS4 and the index primer.

What is claimed is:

1. An inline index based on Illumina sequencing, wherein the inline index is a 6-bp DNA sequence, and the inline index has the following characteristics:
i) it has a minimum editing length of 3 bp or less;
ii) it comprises fragments of different bases;
iii) it comprises bases of different laser colors, wherein adjacent bases are different in laser color; and
iv) it is free of ‘AAA’, ‘ACA’, ‘CCC’, ‘CAC’, ‘GGG’, ‘GTG’, ‘TTT’ and ‘TGT’;

2. The inline index of claim 1, wherein the inline index comprises an IS1 inline index, an IS2 inline index and an IS3 inline index corresponding to the IS1 inline index and IS2 inline index.

3. The inline index of claim 2, wherein the IS1 inline index comprises an IS1 sequence and an IS3X′ sequence partially complementary to the IS1 sequence; and
the IS1 sequence and the IS3X′ sequence are ligated by cooling from 95° C. to 12° C. at a rate of 0.1° C./s.

4. The inline index of claim 2, wherein the IS2 inline index comprises an IS2 sequence and an IS3Y′ sequence partially complementary to the IS2 sequence; and the IS2 sequence and the IS3Y′ sequence are ligated by cooling from 95° C. to 12° C. at a rate of 0.1° C./s.

5. A method of constructing a DNA library labeled by the inline index of claim 2, comprising:
(1) breaking a DNA sequence of a biological sample to obtain a DNA fragment;
(2) repairing a blunt end of the DNA fragment;
(3) ligating an adapter of the IS1 inline index to a 5′ end of the DNA fragment;
and ligating an adapter of the IS2 inline index to a 3′ end of the DNA fragment;
wherein the adapter of the IS1 inline index is inside a binding site of a primer IS7, and the adapter of the IS2 inline index is inside a binding site of a primer IS8;
(4) repairing a gap of the DNA fragment and extending the repaired DNA fragment; and
(5) subjecting the extended DNA fragment to PCR amplification in the use of the primers IS7 and IS8 to produce the DNA library.


SEQUENCE LISTING (95 sequences)

SEQ ID NO: 1 - DNA, 39 bp, Artificial Sequence
  synthetic
acactctttccctacacgacgctcttccgatcttctgcc

SEQ ID NO: 2 - DNA, 39 bp, Artificial Sequence
  synthetic
acactctttccctacacgacgctcttccgatctgtctct

SEQ ID NO: 3 - DNA, 39 bp, Artificial Sequence
  synthetic
acactctttccctacacgacgctcttccgatctatattg

SEQ ID NO: 4 - DNA, 39 bp, Artificial Sequence
  synthetic
acactctttccctacacgacgctcttccgatcttggaag

SEQ ID NO: 5 - DNA, 39 bp, Artificial Sequence
  synthetic
acactctttccctacacgacgctcttccgatcttctagt

SEQ ID NO: 6 - DNA, 39 bp, Artificial Sequence
  synthetic
acactctttccctacacgacgctcttccgatctagagta

SEQ ID NO: 7 - DNA, 39 bp, Artificial Sequence
  synthetic
acactctttccctacacgacgctcttccgatctggccaa

SEQ ID NO: 8 - DNA, 39 bp, Artificial Sequence
  synthetic
acactctttccctacacgacgctcttccgatcttatctc

SEQ ID NO: 9 - DNA, 39 bp, Artificial Sequence
  synthetic
acactctttccctacacgacgctcttccgatctttatgc

SEQ ID NO: 10 - DNA, 39 bp, Artificial Sequence
  synthetic
acactctttccctacacgacgctcttccgatctagttgg

SEQ ID NO: 11 - DNA, 39 bp, Artificial Sequence
  synthetic
acactctttccctacacgacgctcttccgatctgtcaag

SEQ ID NO: 12 - DNA, 39 bp, Artificial Sequence
  synthetic
acactctttccctacacgacgctcttccgatctcagcaa

SEQ ID NO: 13 - DNA, 39 bp, Artificial Sequence
  synthetic
acactctttccctacacgacgctcttccgatcttcgccg

SEQ ID NO: 14 - DNA, 39 bp, Artificial Sequence
  synthetic
acactctttccctacacgacgctcttccgatctctaaga

SEQ ID NO: 15 - DNA, 39 bp, Artificial Sequence
  synthetic
acactctttccctacacgacgctcttccgatctccgctt

SEQ ID NO: 16 - DNA, 39 bp, Artificial Sequence
  synthetic
acactctttccctacacgacgctcttccgatctaagtta

SEQ ID NO: 17 - DNA, 39 bp, Artificial Sequence
  synthetic
acactctttccctacacgacgctcttccgatctggtacc

SEQ ID NO: 18 - DNA, 39 bp, Artificial Sequence
  synthetic
acactctttccctacacgacgctcttccgatctccaggt

SEQ ID NO: 19 - DNA, 39 bp, Artificial Sequence
  synthetic
acactctttccctacacgacgctcttccgatctaatcga

SEQ ID NO: 20 - DNA, 39 bp, Artificial Sequence
  synthetic
acactctttccctacacgacgctcttccgatctaacgca

SEQ ID NO: 21 - DNA, 39 bp, Artificial Sequence
  synthetic
acactctttccctacacgacgctcttccgatctgacgac

SEQ ID NO: 22 - DNA, 39 bp, Artificial Sequence
  synthetic
acactctttccctacacgacgctcttccgatctcgcgct

SEQ ID NO: 23 - DNA, 39 bp, Artificial Sequence
  synthetic
acactctttccctacacgacgctcttccgatctccgtag

SEQ ID NO: 24 - DNA, 39 bp, Artificial Sequence
  synthetic
acactctttccctacacgacgctcttccgatctgtaatc

SEQ ID NO: 25 - DNA, 40 bp, Artificial Sequence
  synthetic
gtgactggagttcagacgtgtgctcttccgatctgacctt

SEQ ID NO: 26 - DNA, 40 bp, Artificial Sequence
  synthetic
gtgactggagttcagacgtgtgctcttccgatcttcataa

SEQ ID NO: 27 - DNA, 40 bp, Artificial Sequence
  synthetic
gtgactggagttcagacgtgtgctcttccgatctcaagag

SEQ ID NO: 28 - DNA, 40 bp, Artificial Sequence
  synthetic
gtgactggagttcagacgtgtgctcttccgatctcgatca

SEQ ID NO: 29 - DNA, 40 bp, Artificial Sequence
  synthetic
gtgactggagttcagacgtgtgctcttccgatctttgatt

SEQ ID NO: 30 - DNA, 40 bp, Artificial Sequence
  synthetic
gtgactggagttcagacgtgtgctcttccgatcttccgag

SEQ ID NO: 31 - DNA, 40 bp, Artificial Sequence
  synthetic
gtgactggagttcagacgtgtgctcttccgatctcctgaa

SEQ ID NO: 32 - DNA, 40 bp, Artificial Sequence
  synthetic
gtgactggagttcagacgtgtgctcttccgatctattctt

SEQ ID NO: 33 - DNA, 40 bp, Artificial Sequence
  synthetic
gtgactggagttcagacgtgtgctcttccgatctgcgact

SEQ ID NO: 34 - DNA, 40 bp, Artificial Sequence
  synthetic
gtgactggagttcagacgtgtgctcttccgatctggcttc

SEQ ID NO: 35 - DNA, 40 bp, Artificial Sequence
  synthetic
gtgactggagttcagacgtgtgctcttccgatctaatacg

SEQ ID NO: 36 - DNA, 40 bp, Artificial Sequence
  synthetic
gtgactggagttcagacgtgtgctcttccgatcttacggt

SEQ ID NO: 37 - DNA, 40 bp, Artificial Sequence
  synthetic
gtgactggagttcagacgtgtgctcttccgatctaccgtc

SEQ ID NO: 38 - DNA, 40 bp, Artificial Sequence
  synthetic
gtgactggagttcagacgtgtgctcttccgatctagaagc

SEQ ID NO: 39 - DNA, 40 bp, Artificial Sequence
  synthetic
gtgactggagttcagacgtgtgctcttccgatctcatagc

SEQ ID NO: 40 - DNA, 40 bp, Artificial Sequence
  synthetic
gtgactggagttcagacgtgtgctcttccgatctaggctc

SEQ ID NO: 41 - DNA, 40 bp, Artificial Sequence
  synthetic
gtgactggagttcagacgtgtgctcttccgatctctgcgg

SEQ ID NO: 42 - DNA, 40 bp, Artificial Sequence
  synthetic
gtgactggagttcagacgtgtgctcttccgatctctcggc

SEQ ID NO: 43 - DNA, 40 bp, Artificial Sequence
  synthetic
gtgactggagttcagacgtgtgctcttccgatctgattag

SEQ ID NO: 44 - DNA, 40 bp, Artificial Sequence
  synthetic
gtgactggagttcagacgtgtgctcttccgatctagatat

SEQ ID NO: 45 - DNA, 40 bp, Artificial Sequence
  synthetic
gtgactggagttcagacgtgtgctcttccgatcttggtcc

SEQ ID NO: 46 - DNA, 40 bp, Artificial Sequence
  synthetic
gtgactggagttcagacgtgtgctcttccgatctgttccg

SEQ ID NO: 47 - DNA, 40 bp, Artificial Sequence
  synthetic
gtgactggagttcagacgtgtgctcttccgatctaagaac

SEQ ID NO: 48 - DNA, 19 bp, Artificial Sequence
  synthetic
ggcagaagatcggaagagc

SEQ ID NO: 49 - DNA, 19 bp, Artificial Sequence
  synthetic
agagacagatcggaagagc

SEQ ID NO: 50 - DNA, 19 bp, Artificial Sequence
  synthetic
caatatagatcggaagagc

SEQ ID NO: 51 - DNA, 19 bp, Artificial Sequence
  synthetic
cttccaagatcggaagagc

SEQ ID NO: 52 - DNA, 19 bp, Artificial Sequence
  synthetic
actagaagatcggaagagc

SEQ ID NO: 53 - DNA, 19 bp, Artificial Sequence
  synthetic
tactctagatcggaagagc

SEQ ID NO: 54 - DNA, 19 bp, Artificial Sequence
  synthetic
ttggccagatcggaagagc

SEQ ID NO: 55 - DNA, 19 bp, Artificial Sequence
  synthetic
gagataagatcggaagagc

SEQ ID NO: 56 - DNA, 19 bp, Artificial Sequence
  synthetic
gcataaagatcggaagagc

SEQ ID NO: 57 - DNA, 19 bp, Artificial Sequence
  synthetic
ccaactagatcggaagagc

SEQ ID NO: 58 - DNA, 19 bp, Artificial Sequence
  synthetic
cttgacagatcggaagagc

SEQ ID NO: 59 - DNA, 19 bp, Artificial Sequence
  synthetic
ttgctgagatcggaagagc

SEQ ID NO: 60 - DNA, 19 bp, Artificial Sequence
  synthetic
cggcgaagatcggaagagc

SEQ ID NO: 61 - DNA, 19 bp, Artificial Sequence
  synthetic
tcttagagatcggaagagc

SEQ ID NO: 62 - DNA, 19 bp, Artificial Sequence
  synthetic
aagcggagatcggaagagc

SEQ ID NO: 63 - DNA, 19 bp, Artificial Sequence
  synthetic
taacttagatcggaagagc

SEQ ID NO: 64 - DNA, 19 bp, Artificial Sequence
  synthetic
ggtaccagatcggaagagc

SEQ ID NO: 65 - DNA, 19 bp, Artificial Sequence
  synthetic
acctggagatcggaagagc

SEQ ID NO: 66 - DNA, 19 bp, Artificial Sequence
  synthetic
tcgattagatcggaagagc

SEQ ID NO: 67 - DNA, 19 bp, Artificial Sequence
  synthetic
tgcgttagatcggaagagc

SEQ ID NO: 68 - DNA, 19 bp, Artificial Sequence
  synthetic
gtcgtcagatcggaagagc

SEQ ID NO: 69 - DNA, 19 bp, Artificial Sequence
  synthetic
agcgcgagatcggaagagc

SEQ ID NO: 70 - DNA, 19 bp, Artificial Sequence
  synthetic
ctacggagatcggaagagc

SEQ ID NO: 71 - DNA, 19 bp, Artificial Sequence
  synthetic
gattacagatcggaagagc

SEQ ID NO: 72 - DNA, 19 bp, Artificial Sequence
  synthetic
aaggtcagatcggaagagc

SEQ ID NO: 73 - DNA, 19 bp, Artificial Sequence
  synthetic
ttatgaagatcggaagagc

SEQ ID NO: 74 - DNA, 19 bp, Artificial Sequence
  synthetic
ctcttgagatcggaagagc

SEQ ID NO: 75 - DNA, 19 bp, Artificial Sequence
  synthetic
tgatcgagatcggaagagc

SEQ ID NO: 76 - DNA, 19 bp, Artificial Sequence
  synthetic
aatcaaagatcggaagagc

SEQ ID NO: 77 - DNA, 19 bp, Artificial Sequence
  synthetic
ctcggaagatcggaagagc

SEQ ID NO: 78 - DNA, 19 bp, Artificial Sequence
  synthetic
ttcaggagatcggaagagc

SEQ ID NO: 79 - DNA, 19 bp, Artificial Sequence
  synthetic
aagaatagatcggaagagc

SEQ ID NO: 80 - DNA, 19 bp, Artificial Sequence
  synthetic
agtcgcagatcggaagagc

SEQ ID NO: 81 - DNA, 19 bp, Artificial Sequence
  synthetic
gaagccagatcggaagagc

SEQ ID NO: 82 - DNA, 19 bp, Artificial Sequence
  synthetic
cgtattagatcggaagagc

SEQ ID NO: 83 - DNA, 19 bp, Artificial Sequence
  synthetic
accgtaagatcggaagagc

SEQ ID NO: 84 - DNA, 19 bp, Artificial Sequence
  synthetic
gacggtagatcggaagagc

SEQ ID NO: 85 - DNA, 19 bp, Artificial Sequence
  synthetic
gcttctagatcggaagagc

SEQ ID NO: 86 - DNA, 19 bp, Artificial Sequence
  synthetic
gctatgagatcggaagagc

SEQ ID NO: 87 - DNA, 19 bp, Artificial Sequence
  synthetic
gagcctagatcggaagagc

SEQ ID NO: 88 - DNA, 19 bp, Artificial Sequence
  synthetic
ccgcagagatcggaagagc

SEQ ID NO: 89 - DNA, 19 bp, Artificial Sequence
  synthetic
gccgagagatcggaagagc

SEQ ID NO: 90 - DNA, 19 bp, Artificial Sequence
  synthetic
ctaatcagatcggaagagc

SEQ ID NO: 91 - DNA, 19 bp, Artificial Sequence
  synthetic
atatctagatcggaagagc

SEQ ID NO: 92 - DNA, 19 bp, Artificial Sequence
  synthetic
ggaccaagatcggaagagc

SEQ ID NO: 93 - DNA, 19 bp, Artificial Sequence
  synthetic
cggaacagatcggaagagc

SEQ ID NO: 94 - DNA, 19 bp, Artificial Sequence
  synthetic
acgtacagatcggaagagc

SEQ ID NO: 95 - DNA, 19 bp, Artificial Sequence
  synthetic
gttcttagatcggaagagc